(19) United States
(12) Patent Application Publication
(10) Pub. No.: US 2018/0023049 A1
(43) Pub. Date: Jan. 25, 2018

(54) SYNTHETIC PEPTIDE HYDROGEL FORMULATIONS FOR USE AS EXTRACELLULAR MATRIX

(71) Applicants: Karl Patrick Gilbert, Danvers, MA (US); 3-D MATRIX, LTD., Tokyo (JP)

(72) Inventors: Eun Seok Gil, Acton, MA (US); Karl Patrick Gilbert, Danvers, MA (US)

(21) Appl. No.: 15/546,980

(22) PCT Filed: Feb. 25, 2016
(86) PCT No.: PCT/US16/19578

§ 371 Date: Jul. 27, 2017

Related U.S. Application Data
(60) Provisional application No. 62/120,783, filed on Feb. 25, 2015.

(51) Int. Cl. C12N 5/00

(52) U.S. Cl. CPC C12N 5/0068; C12N 5/0062; C12N 2533/50; C12N 2539/00

(57) ABSTRACT

Synthetic peptide hydrogel solutions having a pH level of about 3.5 or less and a tonicity within an isotonic osmolality range for use in cell culture experimentation. Related kits and cell culture methods are also disclosed.

### CROSS-REFERENCE TO RELATED APPLICATION

This application claims the benefit of priority to U.S. Provisional Patent Application Ser. No. 62/120,783, filed on Feb. 25, 2015 and titled “SYNTHETIC PEPTIDE HYDROGEL FORMULATIONS FOR USE AS EXTRACELLULAR MATRIX,” the entire disclosure of which is hereby incorporated herein by reference in its entirety for all purposes.

### FIELD OF THE TECHNOLOGY

One or more aspects relate to self-assembling peptide hydrogels that may be used in various medical, research, and industrial applications and, more specifically to formulated hydrogels that may be used in cell culture experimentation.

### BACKGROUND

Cell culture experiments generally involve growing cells on a substrate in a well of a well plate under controlled conditions. The substrate or matrix generally provides support and promotes cell attachment and growth. Media is changed regularly to provide fresh nutrients. Three-dimensional (3D) cell culture is an emerging platform technology that allows for increased cell proliferation, differentiation, and function.

### SUMMARY

In accordance with one or more aspects, a cell culture kit may comprise a synthetic peptide hydrogel solution, a dilution solution, and instructions to: adjust a concentration of the synthetic peptide hydrogel solution with the dilution solution while maintaining its tonicity at a plasma osmolality level and its pH at a level of about 3.5 or less, and conduct a cell culture experiment with the diluted synthetic peptide hydrogel solution.

In some aspects, the synthetic peptide hydrogel comprises: RADARADARADARADA (RADA16). The synthetic peptide hydrogel solution may be formulated such that a pH level of the synthetic peptide hydrogel solution is about 3.5 or less, and a tonicity of the synthetic peptide hydrogel solution is at a plasma osmolality level prior to dilution. The kit may further comprise a cocktail solution formulated to adjust the pH level of the synthetic peptide hydrogel solution to about 3.5 or less, and the tonicity of the synthetic peptide hydrogel solution to the plasma osmolality level prior to dilution. At least one of the cocktail solution and the dilution solution may comprise one or more isotonic agents to control tonicity. The one or more isotonic agents may include salts, sugars, and mixtures thereof. At least one of the cocktail solution and the dilution solution may comprise one or more alkali salts or acidic salts to control pH. In some aspects, the cocktail solution may be hypertonic and have a pH level of between about 1 and about 14.

In some aspects, the kit may further comprise at least one of: a source of cells to be cultured, a cell culture medium, and an isotonic buffer solution. The isotonic buffer solution may comprise PBS, saline, a sugar-based isotonic agent including but not limited to sucrose, or a cell culture medium including but not limited to DMEM. The kit may further comprise a washing solution. The instructions may further provide direction to isolate cultured cells with the washing solution, and to subject the isolated cells to cellular or molecular characterization. In some aspects, the instructions may be directed to a 3-D cell culture protocol in which the plurality of cells is suspended in buffer solution. In other aspects, the instructions may be directed to a 2-D cell culture protocol. The instructions may provide further direction to select the plasma osmolality level based on a target cell type and to use the cocktail solution to achieve the plasma osmolality level in the synthetic peptide hydrogel solution.

In accordance with one or more aspects, a cell culture method may comprise maintaining a pH level of a synthetic peptide hydrogel solution at about 3.5 or less, maintaining a tonicity of the synthetic peptide hydrogel solution at a plasma osmolality level, diluting the peptide hydrogel solution to a predetermined concentration, culturing cells in the peptide hydrogel solution for a predetermined period of time, and isolating the cultured cells.

In some aspects, the method may further comprise subjecting the isolated cells to molecular characterization involving flow cytometry analysis and image observation, cell blotting, or polymerase chain reaction (PCR) testing. The synthetic peptide hydrogel solution may comprise RADARADARADARADA (RADA16). The culturing step may be directed to a 3-D cell culture experiment. In other aspects, the culturing step may be directed to a 2-D cell culture experiment. The culturing step may be directed to recruiting cells into the hydrogels from surrounding cells in vitro or in vivo. The culturing step may be directed to an in vitro cell culture experiment, an ex vivo cell culture experiment, or an in vivo animal or human experiment. In some aspects, the culturing step may involve combination with drugs, cytokines, growth factors, peptides, and/or proteins to improve cell culture and drive cell growth and differentiation. The method may further comprise a step of selecting the plasma osmolality level based on a target species or a target cell type of cell culture experimentation. In some aspects, the tonicity may be in a range of about 260 to about 360 mOsm/L.

In accordance with one or more aspects, a synthetic peptide hydrogel solution may have a pH level of about 1.5 to about 3.5, and a tonicity at plasma osmolality in a range of about 260 to about 360 mOsm/L with respect to a target species and a target cell type.

In some aspects, the target species may be selected from the group consisting of but not limited to: mouse, rat, swine, rabbit, bovine, human, insect, bacteria, and plant. The target cell type may be selected from the group consisting of but not limited to: fibroblast, stem cells, epithelial cells, endothelial cells, neural cells, cardiac cells, kidney cells, blood cells, muscle cells, pancreatic cell, immune cells, dendritic cells, epidermal cells, cancer cells, astrocytes, adipocytes, hepatic cells, osteoblasts, and chondrocytes. The synthetic peptide hydrogel solution may comprise: RADARADARADARADA (RADA16). The synthetic peptide hydrogel solution may be characterized by a storage modulus of at least 5 Pa at 0.5% concentration when measured at 1 rad/sec and 1 Pa of stress. The synthetic peptide hydrogel solution may comprise one or more isotonic agents including salts, sugars, and mixtures thereof to control the tonicity, and one or more alkali salts or acidic salts to control the pH level.

Still other aspects, embodiments, and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. The accompanying drawings are included to provide illustration and a further understanding of the various aspects and embodiments, and are incorporated in and constitute a part of this specification. The drawings, together with the remainder of the specification, serve to explain principles and operations of the described and claimed aspects and embodiments.

### BRIEF DESCRIPTION OF THE DRAWINGS

The accompanying drawings are not intended to be drawn to scale. For purposes of clarity, not every component may be labeled. In the drawings:

FIGS. 1-12 relate to embodiments discussed in the accompanying Example.

### DETAILED DESCRIPTION

In accordance with one or more embodiments, self-assembling peptide hydrogels may be used as a scaffold for cell culture in various applications. PuraMatrix® peptide hydrogel (hereinafter “PuraMatrix®”), commercially available from 3-D Matrix Co., Ltd., for example, is a synthetic, 16-amino acid polypeptide with a repeating sequence of arginine, alanine, and aspartic acid, or RADARADARADARADA (RADA16). PuraMatrix® is known to self-assemble to form a hydrogel under physiological conditions and can be utilized for both cell culture research in vitro as well as various biomedical applications in vivo. Other relevant non-limiting synthetic peptide sequences may be represented by self-assembling peptides having the repeating sequence of lysine, leucine, and aspartic acid (Lys-Leu-Asp (KLD)), and such peptide sequences are represented by (KLD)p, wherein p=2-50. Still other relevant non-limiting synthetic peptide sequences may be represented by self-assembling peptides having the repeating sequence of isoleucine, glutamic acid, isoleucine and lysine (Ile-Glu-Ile-Lys (IEIK)), and such peptide sequences are represented by (IEIK)p, wherein p=2-50. In some non-limiting embodiments, peptide hydrogels such as those disclosed in International Patent Application Publication No. WO2015/138514 titled “SELF-ASSEMBLING PEPTIDE COMPOSITIONS” and assigned to 3-D Matrix, Ltd., which is hereby incorporated herein by reference in its entirety for all purposes, may be formulated. In at least some embodiments, the standard tonicity of a peptide hydrogel solution, such as RADA16, prior to formulation is hypotonic.

In accordance with one or more embodiments, formulating self-assembling peptide hydrogel solutions may increase the stiffness and/or gelation kinetics of the peptide solutions to beneficially overcome the fragility of peptide hydrogels while adding and changing cell culture media over the peptide solutions. Furthermore, formulation may provide a relatively milder pH environment for cells and tissue during experimentation.

In accordance with one or more embodiments, a target pH level and/or tonicity level may be selected at least in part based on the type of cell or tissue to be used for cell culture experimentation. For example, a pH level of the peptide hydrogel may be adjusted to a level of up to about 3.5, for example, up to a level of about 3.4, for improved cell viability by providing a more gentle, less harsh environment.

With respect to tonicity, the tonicity of a peptide hydrogel solution may be adjusted so as to closely match the plasma osmolality of a target cell type and/or target species. For example, the tonicity of the peptide hydrogel solution may be adjusted based on the plasma osmolality of any given cell type. Tonicity levels may range depending on the type of species and/or the type of cell or tissue to be used for cell culture experimentation. In some non-limiting embodiments, a target tonicity may range from about 260 to about 360 mOsm/L. In at least some non-limiting embodiments, a target tonicity may range from about 290 to about 320 mOsm/L. In some specific non-limiting embodiments, the target tonicity may generally be about 300 mOsm/L. Various techniques and calculations for determining or measuring the plasma osmolality of a cell or tissue and/or the tonicity of a peptide hydrogel solution will be readily apparent to those of ordinary skill in the art. Osmolality may be calculated based on one or more molecular characteristics. In some embodiments, osmolality may be measured using an osmometer such as a freezing point depression osmometer, or a vapor pressure depression osmometer. In at least some embodiments, a plasma osmolality of a target cell or tissue type may first be determined and then the tonicity of a synthetic peptide hydrogel solution may be adjusted to match that plasma osmolality as a target tonicity level.

In accordance with one or more embodiments, the use of various synthetic peptide hydrogel solutions for cell culture applications, including but not limited to PuraMatrix®, may be improved via formulation to provide favorable tonicity and/or pH levels. For example, isotonic and pH-adjusted peptide solutions may be used for cell culture experimentation. In accordance with one or more embodiments, a cocktail solution or another intermediary solution may be used to adjust pH and isotonicity of the peptide solution. The isotonic and pH-adjusted peptide solutions may be subsequently mixed with cells or isotonic buffer solution with or without cells, including but not limited to phosphate-buffered saline (PBS), saline, sucrose solution, and Dulbecco's Modified Eagle's Medium (DMEM) to, for example, maintain their pH level close to 3.5 or less and their tonicity at plasma osmolality. In accordance with one or more embodiments, a dilution solution may be used to adjust the concentration of the peptide solution while maintaining the tonicity and pH level of the isotonic and pH-adjusted peptide solution.

In accordance with one or more embodiments, the prepared peptide hydrogels may be used for 3D cell culture. In accordance with one or more embodiments, the prepared peptide hydrogels may be used for two-dimensional (2D) cell culture on the surface of the peptide hydrogels. Specific 2D and 3D cell culture protocols may be used depending on the peptide solution formulation and some non-limiting protocols are described in the accompanying Example. In accordance with one or more embodiments, the prepared peptide hydrogels may be used for recruiting cells into the hydrogels from surrounding cells in vitro or surrounding tissue in vivo. In accordance with one or more embodiments, the prepared peptide hydrogels can be used for in vivo, in vitro, or ex vivo experiments for scaffolding biomaterials, cell delivery and tissue engineering. In accordance with one or more embodiments, the prepared isotonic and pH-adjusted peptide hydrogels can be mixed with drugs, cytokines, growth factors, and proteins to improve cell culture and drive cell growth and differentiation.

In accordance with one or more embodiments, self-assembling peptide hydrogels such as, for example, PuraMatrix® as formulated herein, may be easily washed out using a peptide hydrogel washing solution, so that cells cultured with peptide hydrogels may be beneficially isolated to perform various molecular level characterizations, including but not limited to flow cytometry analysis and cell image observation, cell blotting, and polymerase chain reaction (PCR) test.

In accordance with one or more embodiments, structural stability of peptide hydrogels when adding cell culture media on the peptide solution may be improved. In accordance with one or more embodiments, a less toxic environment for culturing cells with a milder pH level may be gained.

In accordance with one or more embodiments, a peptide solution may be prepared to maintain it tonicity at plasma osmolality with reference to a target cell type and its pH level at a level of about 1.0 to about 3.5, for example up to about 3.4. The tonicity of peptide solutions may be controlled using isotonic agents, including but not limited to salts such as sodium chloride, sugars such as sucrose and dextrose, and any mixture of isotonic agents such as PBS. The pH level of peptide solution may be adjusted using alkali salts, including but not limited to sodium hydroxide, or acidic salts, including but not limited to hydrochloride. Tonicity and/or pH adjustment may be accomplished with a cocktail or intermediate solution as described herein.

In accordance with one or more embodiments, an isotonic and pH-adjusted peptide solution may be formulated just before cell culture experiment by mixing pure peptide aqueous solution with a cocktail or intermediate solution, to maintain its pH level at a level of between about 1.5 and about 3.5 and its tonicity at plasma osmolality with reference to a target cell type. The tonicity of the cocktail solution may be hypertonic, so that the peptide solution formulated with the cocktail solution may be isotonic at a specific mixing ratio. The tonicity of the cocktail solution may be controlled using isotonic agents, including but not limited to salts such as sodium chloride and potassium chloride, sugars such as sucrose and dextrose, and any mixture of isotonic agents such as PBS. The pH level of the cocktail solution may be adjusted to a level of about 1.0 to about 14.0 so that the peptide solution formulated with the cocktail solution at a specific mixing ratio in turn maintains its pH level at about 1.5 to about 3.5. The pH level of the cocktail solution may be adjusted using alkali salts, including not limited to sodium hydroxide or acidic salts including not limited to hydrochloride.

The isotonic and pH-adjusted peptide solution may be subsequently mixed with cells or isotonic buffer solution with/without cells, including but not limited to PBS, saline, isotonic sucrose solution, and DMEM, to maintain its pH level, for example, close to 3.5 or less and its tonicity at plasma osmolality.

In accordance with one or more embodiments, a dilution solution may be prepared to maintain tonicity at plasma osmolality and a pH level at a level of about 1.0 to about 3.5. The tonicity of the dilution solution may be controlled using isotonic agents, including but not limited to salts such as sodium chloride, sugars such as sucrose and dextrose, and any mixture of isotonic agents such as PBS. The pH level of dilution solution may be adjusted using alkali salts, including not limited to sodium hydroxide and acidic salts including not limited to hydrochloride. The isotonic and pH-adjusted dilution solution may be subsequently mixed with the peptide solution to dilute the peptide solution concentration while maintaining its tonicity at plasma osmolality and its pH level at about 1.0 to about 3.5.

In accordance with one or more embodiments, a peptide hydrogel washing solution may be prepared to maintain the hydrogel tonicity at plasma osmolality and the hydrogel pH level at about 1.5 to about 3.5. The tonicity of the washing solution may be controlled using isotonic agents, including but not limited to salts such as sodium chloride, sugars such as sucrose and dextrose, and any mixture of isotonic agents such as PBS. The pH level of washing solution may be adjusted using alkali salts, including not limited to sodium hydroxide and acidic salts including not limited to hydrochloride. The isotonic and pH-adjusted washing solution may be mixed with the peptide hydrogels, where cells are cultured, to completely dissolve hydrogels and to purely isolate cells. Self-assembling peptide hydrogels such as, for example, PuraMatrix®, may be effectively washed out using a peptide hydrogel washing solution, so that cells cultured with the peptide hydrogels may be beneficially isolated for their molecular level characterization, including but not limited to flow cytometry analysis and image observation, cell blotting, and PCR test.

In accordance with one or more embodiments, a cell culture kit may include a synthetic peptide hydrogel solution. The tonicity and/or pH level of the peptide hydrogel solution may have already been adjusted as described herein such as based on a target cell type. Alternatively, a cocktail or intermediate solution may be provided for this purpose and its composition may vary depending on a target cell type for culture experimentation. The kit may include a dilution solution to achieve a desired peptide hydrogel concentration. The kit may include an isotonic buffer solution and/or a washing solution. The kit may include instructions for performing a 3D or 2D cell culture experiment as described herein, as well as other materials to facilitate a cell culture experiment such as but not limited to a source of cells or tissue, a well plate, a pipette, and cell culture medium.

The function and advantages of these and other embodiments will be more fully understood from the following non-limiting example. The example is intended to be illustrative in nature and is not to be considered as limiting the scope of the embodiments discussed herein.

### Example

The following example illustrates the use of self-assembling peptide hydrogels for cell culture. Six examples of cell culture protocols and related formulations that were used in this example are first described in detail below.

Method #1: Isotonic and pH-adjusted peptide solution and dilution solution may be prepared for this method. The peptide solution may be mixed with cells suspended in PBS (pH 7.4). The formulation protocols of the isotonic and pH-adjusted peptide solution and dilution solution for this method are listed in Table 1. The protocols for 3D and 2D cell culture for this method are shown in Table 2.

TABLE 1
Formulation of the components needed for Method #1

| Compo- | | | |
|---|---|---|---|
| nents | Formulation | pH | Tonicity |
| Peptide | Dissolve 100 mg of PuraMatrix ® powder | 2.5 | Isotonic |
| solution | in 5 ml of water, add 5 mL of 2X PBS, and adjust | | |
| | the pH to 2.5 with NaOH or HCl. The peptide | | |
| | solution will be 1% of PuraMatrix ® | | |
| | isotonic solution at pH 2.5. | | |
| Dilution | Add HCl (1N) to 1X PBS to adjust the pH to | 2.5 | Isotonic |
| solution | 2.5. | | |

TABLE 2
Cell culture protocols for Method #1

| | Protocols | |
|---|---|---|
| 3D cell | 1. | Use the isotonic and pH-adjusted peptide solution and the |
| culture | | dilution solution in Table 1. |
| | 2. | If the peptide solution needs to be more diluted, add the |
| | | dilution solution to the peptide solution in the ratios |
| | | described in Table 13 depending on the final |
| | | peptide concentration and mix them by pipetting up and |
| | | down or vortexing. |
| | 3. | Suspend cells in PBS buffer (pH 7.4) |
| | 4. | Mix the peptide solution or the diluted peptide solution |
| | | with the cell suspended PBS solution in a ratio of 1:1 |
| | | by pipetting up and down or vortexing. |
| | 5. | Place the cell and peptide mixture into well plates or |
| | | transwell insert. |
| | 6. | Add cell culture medium on the top of the mixture. |
| | 7. | As an additional protocol, about 5 to about 50 μL of the |
| | | cell and peptide mixture can be directly pipetted into cell |
| | | culture medium to make a cell-encapsulated hydrogel bead. |
| | 8. | Change medium after 1 hour. |
| 2D cell | 1. | Use the isotonic and pH-adjusted peptide solution and the |
| culture | | dilution solution in Table 1. |
| | 2. | Place the isotonic and pH-adjusted peptide solution into |
| | | well plates or transwell insert. |
| | 3. | If the peptide solution needs to be more diluted, add the |
| | | dilution solution to the peptide solution in the ratios |
| | | described in Table 13 depending on the final peptide |
| | | concentration and mix them by pipetting up and down or |
| | | vortexing. |
| | | Then mix the peptide solution or the diluted peptide |
| | | solution with PBS (pH 7.4) buffer in a ratio of 1:1 by |
| | | pipetting up and down or vortexing. |
| | 4. | Place the diluted isotonic and pH-adjusted peptide solution |
| | | into well plates or transwell insert. |
| | 5. | Add cell culture medium on the top of the peptide |
| | | solution. |
| | 6. | Change medium after 1 hour. |
| | 7. | Add cells suspended in cell culture medium on the top |
| | | of the peptide hydrogel. |

Method #2: Pure aqueous peptide solution, cocktail solution to adjust pH and tonicity of peptide solution, and dilution solution may be prepared for this method. The isotonic and pH-adjusted peptide solution may be mixed with cells suspended in PBS (pH 7.4). The formulation protocols of the pure aqueous peptide solution, the cocktail solution, and the dilution solution for this method are listed in Table 3. The protocols for 3D and 2D cell culture for this method are shown in Table 4.

TABLE 3
Formulation of the components needed for Method #2

| Components | Formulation | pH | Tonicity |
|---|---|---|---|
| Peptide | Dissolve 100 mg of RADA16 powder | 2.2 | Hypotonic |
| solution | in 10 ml of water. The peptide | | (in pure water) |
| | solution will be 1% of | | |
| | PuraMatrix ® aqueous solution | | |
| | at pH 2.2. | | |
| Cocktail | Add HCl (1N) to 4 X PBS to adjust | 2.7 | Hypertonic |
| solution | the pH to 2.7. | | (in 4X PBS) |
| Dilution | Add HCl (1N) to 1X PBS to adjust | 2.5 | Isotonic |
| solution | the pH to 2.5. | | (in 1X PBS) |

TABLE 4
Cell culture protocols for Method #2

| | Protocols | |
|---|---|---|
| 3D cell | 1. | Use the peptide solution, the cocktail solution and the |
| culture | | dilution solution in Table 1. |
| | 2. | Mix the peptide solution with the cocktail solution in a |
| | | ratio of 3:1 by pipetting up and down or vortexing. |
| | 3. | If the peptide solution needs to be more diluted, add the |
| | | dilution solution to the peptide solution in the ratios |
| | | described in Table 13 depending on the final peptide |
| | | concentration and mix them by pipetting up and down or |
| | | vortexing. |
| | 4. | Suspend cells in PBS buffer (pH 7.4) |
| | 5. | Mix the peptide solution or the diluted peptide solution |
| | | with the cell suspended PBS solution in a ratio of 2:1 by |
| | | pipetting up and down or vortexing. |
| | 6. | Place the cell and peptide mixture into well plates or |
| | | transwell insert. |
| | 7. | Add cell culture medium on the top of the mixture. |
| | 8. | As an additional protocol, the cell and peptide mixture |
| | | can be directly pipetted into cell culture medium to make a |
| | | cell-encapsulated hydrogel bead. |
| | 9. | Change medium after 1 hour. |
| 2D cell | 1. | Use the peptide solution, the cocktail solution and the |
| culture | | dilution solution in Table 1. |
| | 2. | Mix the peptide solution with the cocktail solution in a |
| | | ratio of 3:1 by pipetting up and down or vortexing. |
| | 3. | Place the isotonic and pH-adjusted peptide solution into |
| | | well plates or transwell insert. |
| | 4. | If the peptide solution needs to be more diluted, add the |
| | | dilution solution to the peptide solution in the ratios |
| | | described in Table 13 depending on the final peptide |
| | | concentration and mix them by pipetting up and down or |
| | | vortexing. |
| | | Mix the peptide solution or the diluted peptide solution |
| | | with PBS (pH 7.4) buffer in a ratio of 2:1 by pipetting |
| | | up and down or vortexing. Place the diluted isotonic and |
| | | pH-adjusted peptide solution into well plates or transwell |
| | | insert. |
| | 5. | Add cell culture medium on the top of the peptide |
| | | solution. |
| | 6. | Change medium after 1 hour. |
| | 7. | Add cells suspended in cell culture medium on the top of |
| | | the peptide hydrogel. |

Method #3: Isotonic and pH-adjusted peptide solution and dilution solution may be prepared for this method. The peptide solution may be mixed with cells suspended in isotonic sucrose solution (10%). The formulation protocols of the isotonic and pH-adjusted peptide solution and dilution solution for this method are listed in Table 5. The protocols for 3D and 2D cell culture for this method are shown in Table 6.

TABLE 5
Formulation of the components needed for Method #3

| Components | | pH | Tonicity |
|---|---|---|---|
| | Formulation 1 of Method #3 | | |
| Peptide | Dissolve 100 mg of PuraMatrix ® | 3.4 | Isotonic |
| solution | powder in 5 ml of water, add 5 mL | | |
| | of 2X PBS, and adjust the pH to | | |
| | 3.4 with NaOH or HCl. The | | |
| | peptide solution will be 1% of | | |
| | PuraMatrix ® isotonic solution | | |
| | at pH 3.4. | | |
| Dilution | Add HCl (1N) to 1X PBS solution | 3.4 | Isotonic |
| solution | to adjust the pH to 3.4 | | |
| | Formulation 2 of Method #3 | | |
| Peptide | Dissolve 100 mg of PuraMatrix ® | 3.4 | Isotonic |
| solution | powder in 5 ml of water, add 5 mL | | |
| | of 20% sucrose solution, and | | |
| | adjust the pH to 3.4 with NaOH or | | |
| | HCl. The peptide solution will be | | |
| | 1% of PuraMatrix ® isotonic | | |
| | solution at pH 3.4. | | |
| Dilution | Prepare 10% sucrose solution | 5~7 | Isotonic |
| solution | | | |

TABLE 6
Cell culture protocols for Method #3

| | Protocols | |
|---|---|---|
| 3D cell | 1. | Use the isotonic and pH-adjusted peptide solution and the |
| culture | | dilution solution in Table 5. |
| | 2. | If the peptide solution needs to be more diluted, add the |
| | | dilution solution to the peptide solution in the ratios |
| | | described in Table 13 depending on the final peptide |
| | | concentration and mix them by pipetting up and down or |
| | | vortexing. |
| | 3. | Suspend cells in 10% sucrose solution. |
| | 4. | Mix the peptide solution or the diluted peptide solution |
| | | with the cell suspended PBS solution in a ratio of 1:1 by |
| | | pipetting up and down or vortexing. |
| | 5. | Place the cell and peptide mixture into well plates or |
| | | transwell insert. |
| | 6. | Add cell culture medium on the top of the mixture. |
| | 7. | As an additional protocol, 5~50 μL of the cell and peptide |
| | | mixture can be directly pipetted into cell culture medium to |
| | | make a cell-encapsulated hydrogel bead. |
| | 8. | Change medium after 1 hour. |
| 2D cell | 1. | Use the isotonic and pH-adjusted peptide solution and the |
| culture | | dilution solution in Table 5. |
| | 2. | Place the isotonic and pH-adjusted peptide solution into well |
| | | plates or transwell insert. |
| | 3. | If the peptide solution needs to be more diluted, add the |
| | | dilution solution to the peptide solution in the ratios |
| | | described in Table 13 depending on the final peptide |
| | | concentration and mix them by pipetting up and down or |
| | | vortexing. |
| | | Mix the peptide solution or the diluted peptide solution with |
| | | 10% sucrose solution in a ratio of 1:1 by pipetting up and |
| | | down or vortexing. |
| | | Place the diluted isotonic and pH-adjusted peptide solution |
| | | into well plates or transwell insert. |
| | 4. | Add cell culture medium on the top of the peptide solution. |
| | 5. | Change medium after 1 hour. |
| | 6. | Add cells suspended in cell culture medium on the top of |
| | | the peptide hydrogel. |

Method #4: Pure aqueous peptide solution, cocktail solution to adjust pH and tonicity of peptide solution, and dilution solution may be prepared for this method. The isotonic and pH-adjusted peptide solution may be mixed with cells suspended in 10% sucrose solution. The formulation protocols of the pure aqueous peptide solution, the cocktail solution, and the dilution solution for this method are listed in Table 7. The protocols for 3D and 2D cell culture for this method are shown in Table 8.

TABLE 7
Formulation of the components needed for Method #4

| Components | | pH | Tonicity |
|---|---|---|---|
| | Formulation 1 of Method #4 | | |
| Peptide | Dissolve 100 mg of RADA16 | 2.2 | Hypotonic |
| solution | powder in 10 ml of water. The | | (in pure |
| | peptide solution will be 1% of | | water) |
| | PuraMatrix ® aqueous solution | | |
| | at pH 2.2. | | |
| Cocktail | Add HCl (1N) to 4 X PBS to adjust | 6.3 | Hypertonic |
| solution | the pH to 6.3. | | (in 4X PBS) |
| Dilution | Add HCl (1N) to 1X PBS to adjust | 3.4 | Isotonic |
| solution | the pH to 3.4. | | (in 1X PBS) |
| | Formulation 2 of Method #4 | | |
| Peptide | Dissolve 100 mg of RADA16 | 2.2 | Hypotonic |
| solution | powder in 10 ml of water. The | | (in pure |
| | peptide solution will be 1% of | | water) |
| | PuraMatrix ® aqueous solution | | |
| | at pH 2.2. | | |
| Cocktail | Prepare 40% Sucrose solution with | 12~13 | Hypertonic |
| solution | 80 μL/mL of NaOH (1N)) | | (in 4X PBS) |
| Dilution | Prepare 10% sucrose solution | 5~7 | Isotonic |
| solution | | | (in 1X PBS) |

TABLE 8
Cell culture protocols for Method #4

| | Protocols | |
|---|---|---|
| 3D cell | 1. | Use the peptide solution, the cocktail solution and the |
| culture | | dilution solution in Table 7. |
| | 2. | Mix the peptide solution with the cocktail solution in a |
| | | ratio of 3:1 by pipetting up and down or vortexing. |
| | 3. | If the peptide solution needs to be more diluted, add the |
| | | dilution solution to the peptide solution in the ratios |
| | | described in Table 13 depending on the final peptide |
| | | concentration and mix them by pipetting up and down or |
| | | vortexing. |
| | 4. | Suspend cells in 10% sucrose solution. |
| | 5. | Mix the peptide solution or the diluted peptide solution |
| | | with the cell suspended PBS solution in a ratio of 2:1 by |
| | | pipetting up and down or vortexing. |
| | 6. | Place the cell and peptide mixture into well plates or |
| | | transwell insert. |
| | 7. | Add cell culture medium on the top of the mixture. |
| | 8. | As an additional protocol, the cell and peptide mixture can |
| | | be directly pipetted into cell culture medium to make a |
| | | cell-encapsulated hydrogel bead. |
| | 9. | Change medium after 1 hour. |
| 2D cell | 1. | Use the peptide solution, the cocktail solution and the |
| culture | | dilution solution in Table 1. |
| | 2. | Mix the peptide solution with the cocktail solution in a |
| | | ratio of 3:1 by pipetting up and down or vortexing. |
| | 3. | Place the isotonic and pH-adjusted peptide solution into |
| | | well plates or transwell insert. |
| | 4. | If the peptide solution needs to be more diluted, add the |
| | | dilution solution to the peptide solution in the ratios |
| | | described in Table 13 depending on the final peptide |
| | | concentration and mix them by pipetting up and down or |
| | | vortexing. |
| | | Mix the peptide solution or the diluted peptide solution |
| | | with 10% sucrose solution in a ratio of 2:1 by pipetting |
| | | up and down or vortexing. |
| | | Place the diluted isotonic and pH-adjusted peptide |
| | | solution into well plates or transwell insert. |
| | 5. | Add cell culture medium on the top of the peptide |
| | | solution. |
| | 6. | Change medium after 1 hour. |
| | 7. | Add cells suspended in cell culture medium on the top of |
| | | the peptide hydrogel. |

Method #5: Isotonic and pH-adjusted peptide solution and dilution solution may be prepared for this method. The peptide solution may be mixed with cells suspended in DMEM. The formulation protocols of the isotonic and pH-adjusted peptide solution and dilution solution for this method are listed in Table 9. The protocols for 3D and 2D cell culture for this method are shown in Table 10.

TABLE 9
Formulation of the components needed for Method #5

| Components | Formulation | pH | Tonicity |
|---|---|---|---|
| Peptide | Dissolve 100 mg of PuraMatrix ® powder | 2.1 | Isotonic |
| solution | in 5 ml of water, add 5 mL of 2X PBS, and | | |
| | adjust the pH to 2.1 with NaOH or HCl. The | | |
| | peptide solution will be 1% of PuraMatrix ® | | |
| | isotonic solution at pH 2.1. | | |
| Dilution | Add HCl (1N) to 1X PBS to adjust the pH to | 2.1 | Isotonic |
| solution | 2.1. | | |

TABLE 10
Cell culture protocols for Method #5

| | Protocols | |
|---|---|---|
| 3D cell | 1. | Use the isotonic and pH-adjusted peptide solution and the |
| culture | | dilution solution in Table 9. |
| | 2. | If the peptide solution needs to be more diluted, add the |
| | | dilution solution to the peptide solution in the ratios |
| | | described in Table 13 depending on the final peptide |
| | | concentration and mix them by pipetting up and down or |
| | | vortexing. |
| | 3. | Suspend cells in DMEM. |
| | 4. | Mix the peptide solution or the diluted peptide solution |
| | | with the cell suspended DMEM in a ratio of 1:1 by |
| | | pipetting up and down or vortexing. |
| | 5. | Place the cell and peptide mixture into well plates or |
| | | transwell insert. |
| | 6. | Add cell culture medium on the top of the mixture. |
| | 7. | As an additional protocol, 5~50 μL of the cell and peptide |
| | | mixture can be directly pipetted into cell culture medium |
| | | to make a cell-encapsulated hydrogel bead. |
| | 8. | Change medium after 1 hour. |
| 2D cell | 1. | Use the isotonic and pH-adjusted peptide solution and the |
| culture | | dilution solution in Table 9. |
| | 2. | Place the isotonic and pH-adjusted peptide solution into |
| | | well plates or transwell insert. |
| | 3. | If the peptide solution needs to be more diluted, add the |
| | | dilution solution to the peptide solution in the ratios |
| | | described in Table 13 depending on the final peptide |
| | | concentration and mix them by pipetting up and down or |
| | | vortexing. |
| | | Mix the peptide solution or the diluted peptide solution |
| | | with DMEM in a ratio of 1:1 by pipetting up and down |
| | | or vortexing. |
| | | Place the diluted isotonic and pH-adjusted peptide |
| | | solution into well plates or transwell insert. |
| | 4. | Add cell culture medium on the top of the peptide |
| | | solution. |
| | 5. | Change medium after 1 hour. |
| | 6. | Add cells suspended in cell culture medium on the top of |
| | | the peptide hydrogel. |

Method #6: Pure aqueous peptide solution, cocktail solution to adjust pH and tonicity of peptide solution, and dilution solution may be prepared for this method. The isotonic and pH-adjusted peptide solution may be mixed with cells suspended in DMEM. The formulation protocols of the pure aqueous peptide solution, the cocktail solution, and the dilution solution for this method are listed in Table 11. The protocols for 3D and 2D cell culture for this method are shown in Table 12.

TABLE 11
Formulation of the components needed for Method #6

| Components | Formulation protocols | pH | Tonicity |
|---|---|---|---|
| Peptide | Dissolve 100 mg of RADA16 | 2.2 | Hypotonic |
| solution | powder in 10 ml of water. | | (in pure |
| | The peptide solution will | | water) |
| | be 1% of PuraMatrix ® | | |
| | aqueous solution at pH 2.2. | | |
| Cocktail | Add HCl (1N) to 4X PBS to | 1.8 | Hypertonic |
| solution | adjust the pH to 1.8. | | (in 4X |
| | | | PBS) |
| Dilution | Add HCl (1N) to 1X PBS to | 3.4 | Isotonic |
| solution | adjust the pH to 2.1. | | (in 1X |
| | | | PBS) |

TABLE 12
Cell culture protocols for Method #6

| | Protocols | |
|---|---|---|
| 3D cell | 1. | Use the peptide solution, the cocktail solution and the |
| culture | | dilution solution in Table 11. |
| | 2. | Mix the peptide solution with the cocktail solution in a |
| | | ratio of 3:1 by pipetting up and down or vortexing. |
| | 3. | If the peptide solution needs to be more diluted, add the |
| | | dilution solution to the peptide solution in the ratios |
| | | described in Table 13 depending on the final peptide |
| | | concentration and mix them by pipetting up and down or |
| | | vortexing. |
| | 4. | Suspend cells in DMEM. |
| | 5. | Mix the peptide solution or the diluted peptide solution |
| | | with the cell suspended DMEM in a ratio of 2:1 by |
| | | pipetting up and down or vortexing. |
| | 6. | Place the cell and peptide mixture into well plates or |
| | | transwell insert. |
| | 7. | Add cell culture medium on the top of the mixture. |
| | 8. | As an additional protocol, the cell and peptide mixture |
| | | can be directly pipetted into cell culture medium to make |
| | | a cell-encapsulated hydrogel bead. |
| | 9. | Change medium after 1 hour. |
| 2D cell | 1. | Use the peptide solution, the cocktail solution and the |
| culture | | dilution solution in Table 11. |
| | 2. | Mix the peptide solution with the cocktail solution in a |
| | | ratio of 3:1 by pipetting up and down or vortexing. |
| | 3. | Place the isotonic and pH-adjusted peptide solution into |
| | | well plates or transwell insert. |
| | 4. | If the peptide solution needs to be more diluted, add the |
| | | dilution solution to the peptide solution in the ratios |
| | | described in Table 13 depending on the final peptide |
| | | concentration and mix them by pipetting up and down or |
| | | vortexing. |
| | | Mix the peptide solution or the diluted peptide solution |
| | | with DMEM in a ratio of 2:1 by pipetting up and down |
| | | or vortexing. |
| | | Place the diluted isotonic and pH-adjusted peptide |
| | | solution into well plates or transwell insert. |
| | 5. | Add cell culture medium on the top of the peptide |
| | | solution. |
| | 6. | Change medium after 1 hour. |
| | 7. | Add cells suspended in cell culture medium on the top of |
| | | the peptide hydrogel. |

The mixing ratios used in this Example are presented below in Table 13.

TABLE 13
Mixing ratios of isotonic and pH-adjusted peptide solution to
dilution solution

| | Final peptide | Mixing ratios of isotonic and pH-adjusted |
|---|---|---|
| Methods | concentration | peptide solution to dilution solution |
| # 1-6 | 0.5% | No addition of dilution solution |
| | 0.4% | 4:1 |
| | 0.3% | 3:2 |
| | 0.25% | 1:1 |
| | 0.2% | 2:3 |
| | 0.1% | 1:4 |

### Rheological Properties:

In accordance with one or more embodiments, rheological properties may vary with the preparation method of the peptide hydrogels for cell culture.

The rheological properties of peptides were evaluated at the concentration of 0.5% using a rheometer (DHR-1, TA Instruments) with 20 mm plates. Peptide solution (200 μL) was placed on the rheometer plate and the moduli were measured at 25° C. with the plates placed at a measuring geometry gap of 500 μm. Measurements were performed after 2 minutes of relaxation time at 25° C. Frequency sweep tests were performed at 1 rad/sec˜10 red/sec of oscillation stress with stress at 1 Pa.

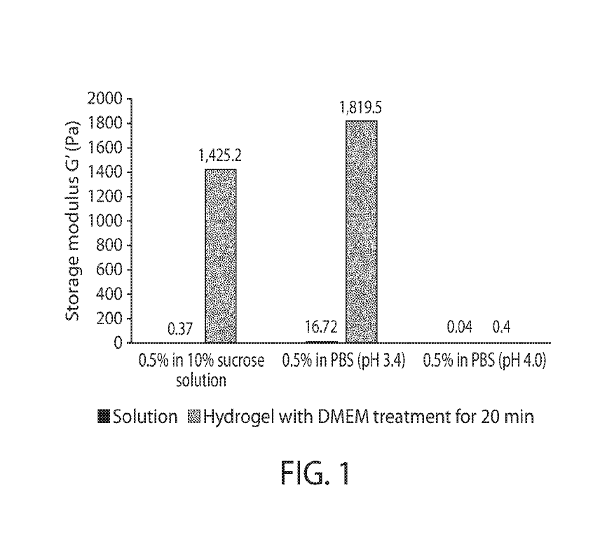

The rheology results are shown in FIG. 1 for PuraMatrix® prepared in three different isotonic buffers. Specifically, FIG. 1 presents storage moduli data of isotonic and pH-adjusted PuraMatrix®. PuraMatrix® solutions (0.5%) were prepared in 1) 10% sucrose solution, 2) 1×PBS at pH 3.4, and 3) 1×PBS at pH 4.0. Gelation was induced by treating PuraMatrix® solutions with DMEM for 20 min.

The peptide solutions were treated with DMEM for 20 min to induce gelation. The storage moduli of three samples at 1 rad/sec were shown in FIG. 1. As the first sample, an isotonic PuraMatrix® solution was formulated with 10% sucrose as a control because this formation has been used for a common cell culture method as demonstrated in the PuraMatrix® Guidelines for Use (BD/Corning website). As the second sample, an isotonic PuraMatrix® solution was prepared with PBS at pH 3.4 just below the isoelectronic point (pI) of PuraMatrix®. As the third sample, an isotonic PuraMatrix® solution was made with PBS at pH 4.0 at which PuraMatrix® is at its isoelectronic point (pI).

Isoelectronic point (pI) is the pH at which a particular molecule carries no net electrical charge. PuraMarix® has four arginine and four aspartic acid. PuraMatrix® may have an isoelectronic point (pI) from around pH 3.9 to around pH 11.5, because the pKa of aspartic acid in PuraMatrix® is 3.9 and the pKb of arginine in PuraMatrix® is 11.5. That means that there is no net electrical charge between around pH 3.9 and around pH 11.5, where the solubility of PuraMatrix® becomes low. Usually, aqueous PuraMatrix® solution has its pH level at 2.0-2.5. In this study, the aqueous 1% PuraMatrix® solution showed its pH at 2.2. Although isotonic PuraMatrix® formulated in 10% sucrose solution has been used for cell culture experiments as shown in the current PuraMatrix® cell culture protocol, the pH of PuraMatrix® with 10% sucrose was also 2.2. The storage modulus of the Isotonic PuraMatrix® solution with PBS at pH 3.4 was noticeably higher than that of the Isotonic PuraMatrix® solution with 10% sucrose. After the isotonic PuraMatrix® solutions were treated with DMEM for their gelation, the storage modulus of the Isotonic PuraMatrix® with PBS at pH 3.4 was slightly higher than that of Isotonic PuraMatrix® with 10% sucrose. However, the Isotonic PuraMatrix® with PBS at pH 4.0 showed very low storage modulus and did not form a gel with DMEM treatment.

Especially, the stiffness of PuraMatrix® solution is very important to keep the structure stability of peptide hydrogel when cell culture medium is added on the top of the peptide solution. If the stiffness of peptide solution is weak, the peptide solution may be unstable during gelation or washed out with medium because flow force from pipetted medium may destroy the structure stability of weak peptide solution before gelation.

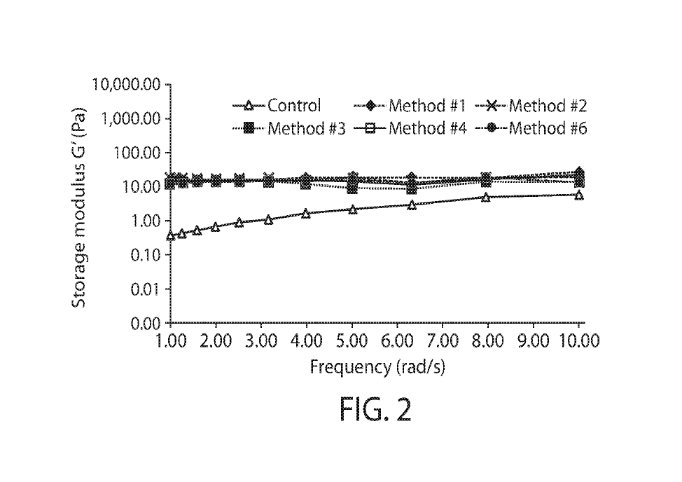

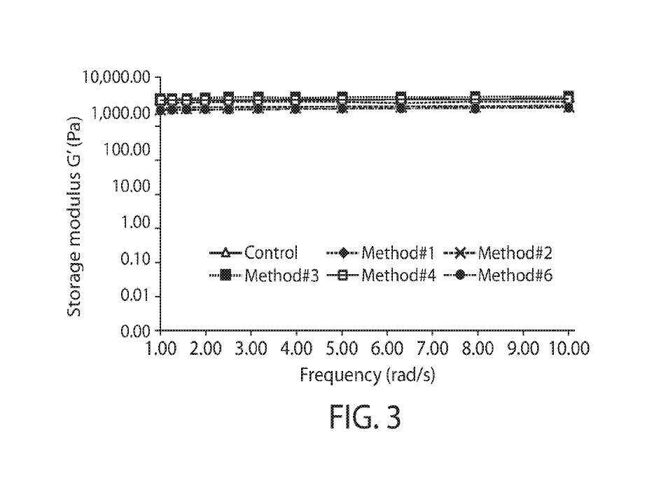

The rheological properties of peptides prepared from the methods #1-6 were evaluated and the results were shown in FIGS. 2 and 3. FIG. 2 presents the rheological property of the 0.5% PuraMatrix® solutions prepared from the methods #1-6. FIG. 3 presents the rheological property of the 0.5% PuraMatrix® hydrogels prepared from the methods #1-6. PuraMatrix® (0.5%) was treated with DMEM for 20 min to form a hydrogel. The stiffness of isotonic PuraMatrix® solution prepared from the methods #1-6 was much higher than that prepared from the control method. However, after these isotonic PuraMatrix® solutions were treated with DMEM to form a gel, the storage moduli of all the hydrogels were similar.

### Gelation Kinetics:

In accordance with one or more embodiments, gelation kinetics of peptides may vary with the preparation method of peptide isotonic solutions. The gelation kinetics of isotonic peptide solutions were evaluated at 0.5% using a rheometer (DHR-1, TA Instruments) with 25 mm plates. Peptide solution (200 μL) was placed on the rheometer plate and gelation kinetics were measured at 25° C. with the plates placed at a measuring geometry gap of 500 μm. Measurements were performed after 2 minutes of relaxation time at 25° C. Time sweep tests were performed at 1 Hz with 0.3% of strain. During the times sweep, 10 mL of DMEM solution was added around peptide solution. A liquid chamber was used for this test to make peptide solution soaked in DMEM. A zero time point was set as the time when DMEM was added to the liquid chamber around PuraMatrix® sample located between two plates during time sweep tests. The modulus was recorded every around 2.5 Seconds.

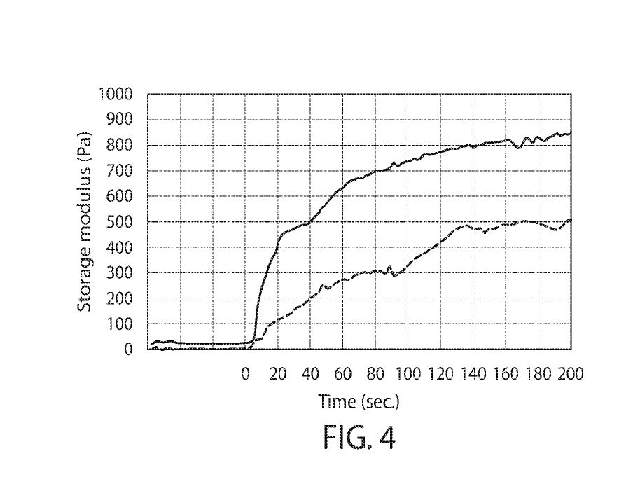

The gelation kinetics results are shown in FIG. 4 for PuraMatrix® solutions prepared from two different isotonic buffers. The dash line and solid line represent gelation kinetics of 0.5% PuraMatrix® prepared in 10% sucrose solution and 0.5% PuraMatrix® prepared in PBS buffer at pH 3.4, respectively. DMEM was added to the liquid chamber around PuraMatrix® sample located between two plates at a zero time point during time sweep tests. As the first sample, an isotonic PuraMatrix® solution was formulated with 10% sucrose as a control because this formation has been used for a common cell culture method as demonstrated in the PuraMatrix® Guidelines for Use (BD/Corning website). As the second sample, an isotonic PuraMatrix® solution was prepared with PBS at pH 3.4 just below the isoelectronic point (pI) of PuraMatrix®. When the peptide solutions were treated with DMEM, the gelation kinetics of the Isotonic PuraMatrix® solution with PBS at pH 3.4 was faster than that of the Isotonic PuraMatrix® solution with 10% sucrose.

The gelation kinetics of isotonic PuraMatrix® solution may be also important to keep the structure stability of peptide hydrogel when cell culture medium is added on peptide solution. If the gelation kinetics of peptide solution is slow, the peptide solution may be easily broken or washed out with added cell culture medium before gelation.

### Structural Stability:

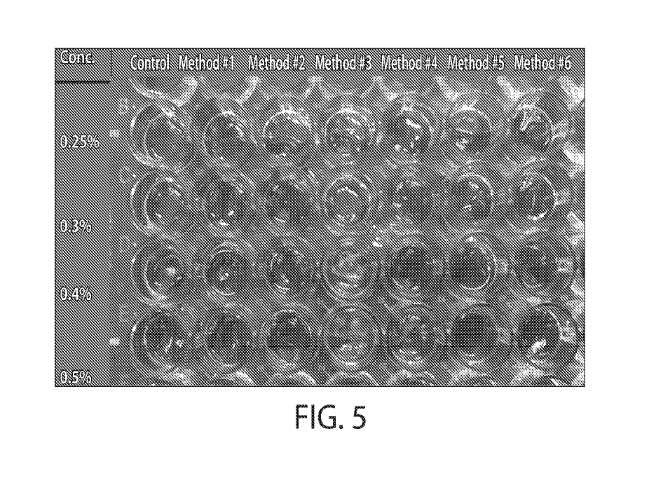

In accordance with one or more embodiments, the structural stability of peptide hydrogels during cell culture may vary with preparation methods of isotonic and pH-adjusted peptide solutions. The structure stability of isotonic peptide hydrogels were visually evaluated using Method #1-6 compared to that of isotonic peptide hydrogel with 10% sucrose as a control which is a common cell culture method as demonstrated in the PuraMatrix® Guidelines for Use (BD/Corning website). Peptide solution (200 μL) was placed on the 48 well plates and DMEM (500 μL) was gently added. After 1 hour, medium was changed with fresh DMEM (500 μL). The images of the hydrogels after removing DMEM were taken on next day. The image for the apparent structure stability of peptide hydrogels is shown in FIG. 5 for the peptide hydrogels prepared from Methods #1-6 and control at different peptide concentration and their evaluations were listed in Table 14. Apparently, the structures of the peptide hydrogels prepared from the methods #1-6 seem more stable than that prepared from the control method. All the peptide hydrogels are apparently transparent.

TABLE 14
Apparent structure stability of hydrogels with different methods.

| | | Pro- | Pro- | Pro- | Pro- | Pro- | Pro- |
|---|---|---|---|---|---|---|---|
| | | tocol | tocol | tocol | tocol | tocol | tocol |
| Conc. | Control | #1 | #2 | #3 | #4 | #5 | #6 |
| 0.25% | Se- | Flat | Severely | Severely | Partially | Partially | Flat |
| | verely | surface | Broken | Broken | broken | broken | surface |
| | Broken | with- | | | with | with | with- |
| | | out | | | rough | rough | out |
| | | break | | | surface | surface | break |
| 0.3% | Se- | Flat | Flat | Severely | Flat | Partially | Flat |
| | verely | surface | surface | Broken | surface | broken | surface |
| | Broken | with- | without | | without | with | with- |
| | | out | break | | break | rough | out |
| | | break | | | | surface | break |
| 0.4% | Se- | Flat | Flat | Flat | Flat | Flat | Flat |
| | verely | surface | surface | surface | surface | surface | surface |
| | Broken | with- | without | without | without | without | with- |
| | | out | break | break | break | break | out |
| | | break | | | | | break |
| 0.5% | Par- | Flat | Flat | Flat | Flat | Flat | Flat |
| | tially | surface | surface | surface | surface | surface | surface |
| | broken | with- | with- | with- | with- | with- | with- |
| | & rough | out | out | out | out | out | out |
| | surface | break | break | break | break | break | break |

### 3D Cell Culture:

In accordance with one or more embodiments, a peptide hydrogel may be used for 3D cell culture. Each peptide solution was prepared at a concentration of 1% and then diluted to concentrations of 0.50% or less following the methods #1-6 or the control method as described in the PuraMatrix® Guidelines for Use (BD/Corning website). NIH 3T3 mouse embryonic fibroblasts or C57 BL/6 Mouse Mesenchymal Stem Cells (mMSCs) were washed and re-suspended in PBS (pH 7.4) for the method #1 and 2, in 10% sucrose for the method #3 and 4, and DMEM for the methods #5 and 6. The cells were re-suspended in each of the concentrations of peptide solutions to a final concentration of 1 million cells/ml. The cell culture medium used is DMEM (with high glucose, L-glutamine, and sodium pyruvate), 1% antibiotic antimyotic solution, and 10% bovine calf serum. The peptide/cell mixture of 100 μL was placed on each 48 well cell culture plate and DMEM of 300 μL was added on the top of the peptide/cell mixture. The protocol was then followed for 3D cell culture in cell culture plates as described in Tables 1-12 for the methods #1-6 and the PuraMatrix® Guidelines for Use (BD/Corning website) for the control method.

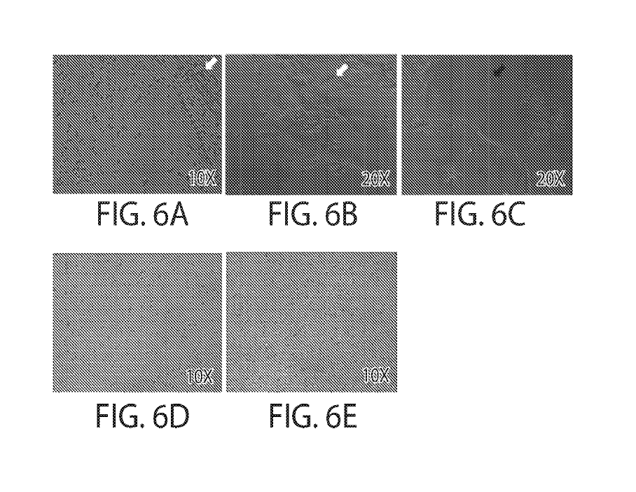

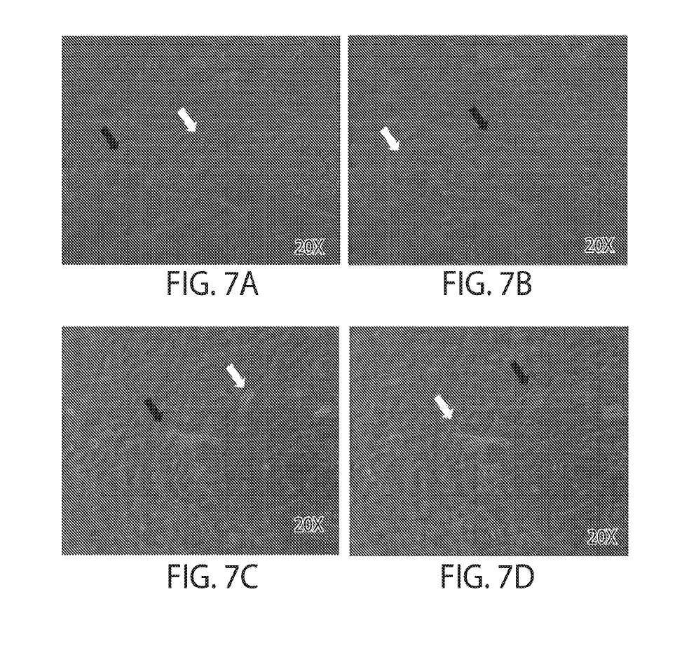

The microscopic images of cells 3D-cultured in the peptide hydrogels prepared from the methods and the control method following the PuraMatrix® Guidelines for Use (BD/Corning website) are shown in FIGS. 6 and 7. Specifically, FIG. 6 presents microscopic images of NIH 3T3 mouse embryonic fibroblasts three-dimensionally cultured in 0.5% PuraMatrix® for 24 hours. (A-C) Control: Cells were cultured with 0.5% PuraMatrix® in 10% sucrose solution following the PuraMatrix® Guidelines for Use (BD/Corning website) (B) Method #1: Cells were cultured with 0.5% PuraMatrix® formulated in PBS at pH 3.4, (E) Method #3: Cells were cultured with 0.5% PuraMatrix® formulated in isotonic sucrose and PBS mixture at pH 3.4. White arrows reveal damaged hydrogel area and black arrows demonstrate a blank plastic bottom due to severe hydrogel damage. FIG. 7 presents microscopic images of NIH 3T3 mouse embryonic fibroblasts and C57 BL/6 Mouse Mesenchymal Stem Cells (mMSCs) three-dimensionally cultured in 0.5% PuraMatrix® using Method #1 for 48 hours. (A-B) NIH 3T3 mouse embryonic fibroblasts and (C-D) C57 BL/6 Mouse Mesenchymal Stem Cells (mMSCs) were cultured with 0.5% PuraMatrix® and formulated in PBS at pH 3.4: two images in (A) and (B) or (C) and (D) were taken at a same position with different focus depth. White arrows point the well-focused cells, while black arrows point the unfocused cells. Therefore, cells are 3-dimensionally embedded in the hydrogels.

All the peptide hydrogels were transparent, so that the 3-D encapsulated cells in the peptide hydrogels could be microscopically observed. However, the peptide hydrogel prepared with cells from the control method showed frequent broken parts, while the peptide hydrogels prepared with cells from the methods #1 and 3 showed clear and consistent matrix visualization over the entire area as shown in FIG. 6. The other hydrogels prepared from the methods #2, 4-6 can be used for 3D cell culture because they are all transparent and have similar stiffness to the hydrogel prepared from method #1 and #3. Cells were well three-dimensionally distributed in the hydrogels with two different cell types as shown in FIG. 7.

### 2D Cell Culture:

In accordance with one or more embodiments, a peptide hydrogel may be used for 2D cell culture. Each peptide solution was prepared at a concentration of 1% and then diluted to concentrations of 0.50% or less following the methods #1-6. The peptide solution of 160 μL was placed on 48 well cell culture plates and kept in 2-8 C for 12 hours. DMEM of 320 μL was added to the top of the gel by slowly pipetting down the sides of the well and the gel was incubated for 20 min at 37° C. in a cell culture incubator and DMEM was carefully removed. The cells were suspended in DMEM of 320 μL to a final concentration of 5000 cells/cm2 or 100,000 cells/cm² and placed on the top of the gel.

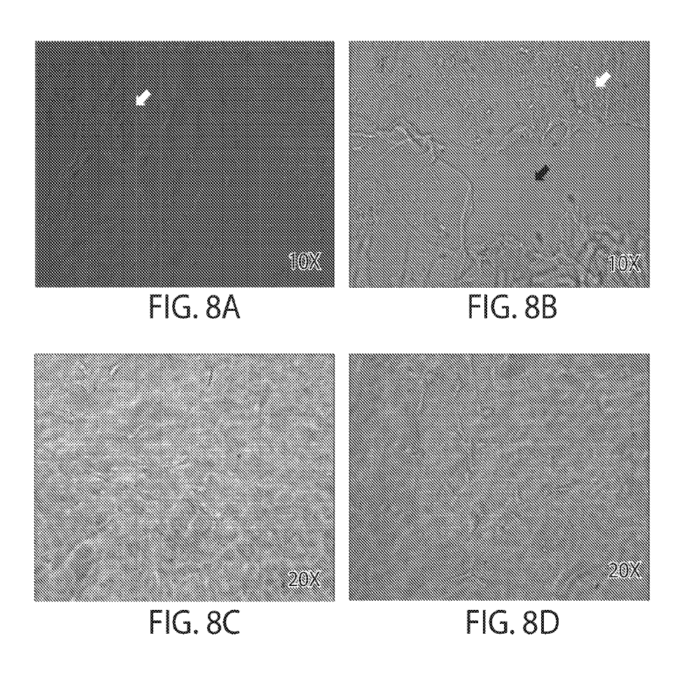

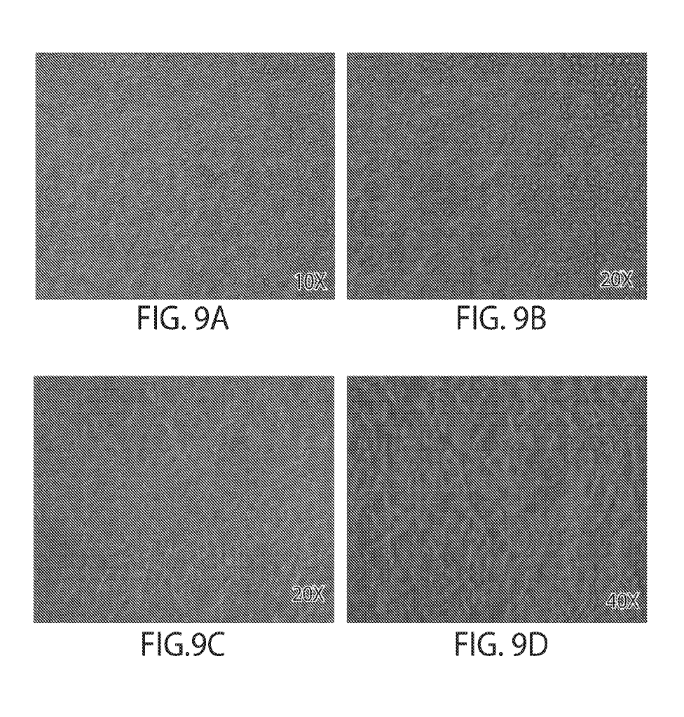

The microscopic images of cells 2-D cultured on the surface of the peptide hydrogels prepared from the methods #1 are shown in FIGS. 8 and 9. Specifically, FIG. 8 presents microscopic images of NIH 3T3 mouse embryonic fibroblasts (A and C) and C57 BL/6 Mouse Mesenchymal Stem Cells (mMSCs) (B and D). (A-B) Control: Cells were cultured with 0.5% PuraMatrix® in 10% sucrose solution following the PuraMatrix® Guidelines for Use (BD/Corning website) for 2 days (C-D) Method #1: Cells were cultured with 0.5% PuraMatrix® formulated in PBS at pH 3.4 for 2 days. Cells were seeded at 5000 cells/cm². White arrows reveal damaged hydrogel area and black arrows demonstrate an opened plastic bottom due to severe hydrogel damage. FIG. 9 presents microscopic images of NIH 3T3 mouse embryonic fibroblasts 2-D cultured in 0.5% PuraMatrix® prepared from the method #1 for 8 hours (A-B) and 4 days (C-D). Cells were seeded at 100,000 cells/cm².

The peptide hydrogels prepared from the Method #1 are transparent, so that the 2-D cultured cells on the peptide hydrogels could be clearly observed as shown in FIG. 8. Cells were well two-dimensionally distributed in the hydrogels with two different cell types. However, the peptide hydrogel prepared with cells from the control method showed frequent broken parts, so that cells were not seeded on the damaged area or went down onto plastic bottom. Cells became confluent on the top of the hydrogel without defects after they were cultured for 4 days as shown in FIG. 9. The other hydrogels prepared from the methods #2-6 can be used for 2D cell culture because they are all transparent and have similar stiffness to the hydrogel prepared from method #1. The seeded cells were evenly distributed on the hydrogel surface and began to be attached on the surface in 8 hours. The cells were well attached and confluent on the gel surface in four days. However, the 0.5% PuraMatrix® prepared from 10% sucrose solution following the PuraMatrix® Guidelines for Use (BD/Corning website) did not generate a flat surface for 2D cell culture due to its weak structure stability during cell culture medium addition on the top of PuraMatrix®.

### Washing:

In accordance with one or more embodiments, a peptide hydrogel may be entirely washed out to isolate encapsulated or attached cells from the peptide hydrogels. Peptide hydrogel washing solution may be prepared with isotonic and pH-adjusted buffers such as 10% sucrose and PBS, whose pH may be adjusted at 1.5-3.5 using acidic salt such as hydrochloride. In this test, the peptide hydrogel washing solution was formulated with PBS at pH 3.0 by adding 1 N hydrochloride in PBS (pH 7.4) buffer. PuraMatrix® 1% solution of 200 μL was placed in 2 mL centrifuge tube and 1 mL of DMEM hydrogels was gently added to induce gelation. After 1 hour, the medium was removed and the gel was broken and washed by pipetting with 1 mL of three different washing solutions: (1) 10% sucrose solution, (2) PBS (pH 7.4), and (3) PBS (pH 3.0). The tube was centrifuged at 1100 rpm for 5 min, the supernatant was removed. The hydrogels were washed and centrifuged again and the remained pellet was re-suspended with PBS buffer (pH 7.4).

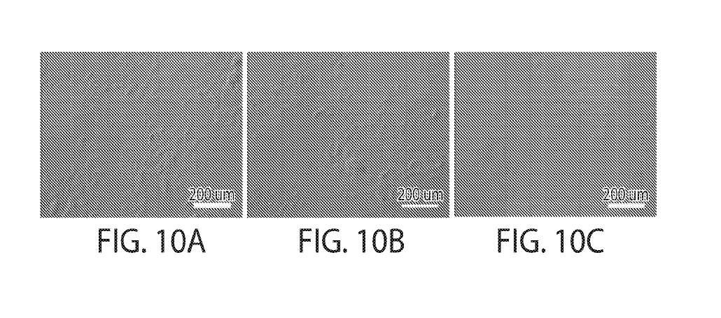

The microscopic images of the peptide hydrogels after being washed twice with the washing buffers are shown in FIG. 10: A) washed with 10% sucrose solution, (B) washed with PBS (pH 7.4), and (C) washed with the peptide hydrogel washing solution (PBS at pH 3.0). The peptide hydrogels were washed twice with 10% sucrose solution, PBS (pH 7.4) and PBS (pH 3.0). 10% sucrose solution and PBS (pH 7.4) did not successfully washed peptide hydrogels showing abundant broken gel particles whose size is around 10˜100 μm. However, the peptide hydrogel washing solution entirely washed out peptide hydrogels.

### Cell Characterization:

In accordance with one or more embodiments, after a peptide hydrogel is entirely washed out, the isolated cells from the peptide hydrogels may be beneficially utilized for the molecular level characterization including but not limited to flow cytometry. The isolated cells washed with the peptide washing solution may be stained with fluorescence dies and tested for flow cytometry without interference of remained peptide hydrogels. Cells were 3-dimensionally cultured in the hydrogels prepared from the methods #1-6 and the control method following the PuraMatrix® Guidelines for Use (BD/Corning website). The cell culture medium used is DMEM (with high glucose, L-glutamine, and sodium pyruvate), 1% antibiotic antimyotic solution, and 10% bovine calf serum. The cultures were done as 3D plates. The cultures were plated with 100 ul of the hydrogel with 1,000,000 cells/ml in 48 well culture plates. 300 μL of cell culture media was added on top of the gels and was changed with fresh 300 μL of cell culture media in 1 hour. Cells were cultured for 24 hours before harvesting cells from the cultures. To isolate the cells from the cultures, 0.500 ml of PBS was added to the wells. The gels were broken by pipetting and added to mini centrifuge tubes. 0.500 ml of PBS was added to the wells for a second wash and that was also added to the centrifuge tubes. The cells and gel were centrifuged at 1100 RPM for 5 min. As an optional process, Trypsin-EDTA can be added and the samples can be incubated for 5 min at 37° C. After supernatant was removed, 1 ml of the PuraMatrix® washing Solution was added. The samples were centrifuged and 1 ml of supernatant was removed. The washing solution was added and the samples were centrifuged again. 1 ml of supernatant was again removed and the cells were re-suspended in 0.500 ml of PBS (pH 7.4). The cells were stained with propidium iodide (Life technologies) following the protocol from Life technologies. Propidium iodide can be used to detect dead cells in a population because it is not permeant to live cells and only stains the nuclear and chromosome of dead cells.

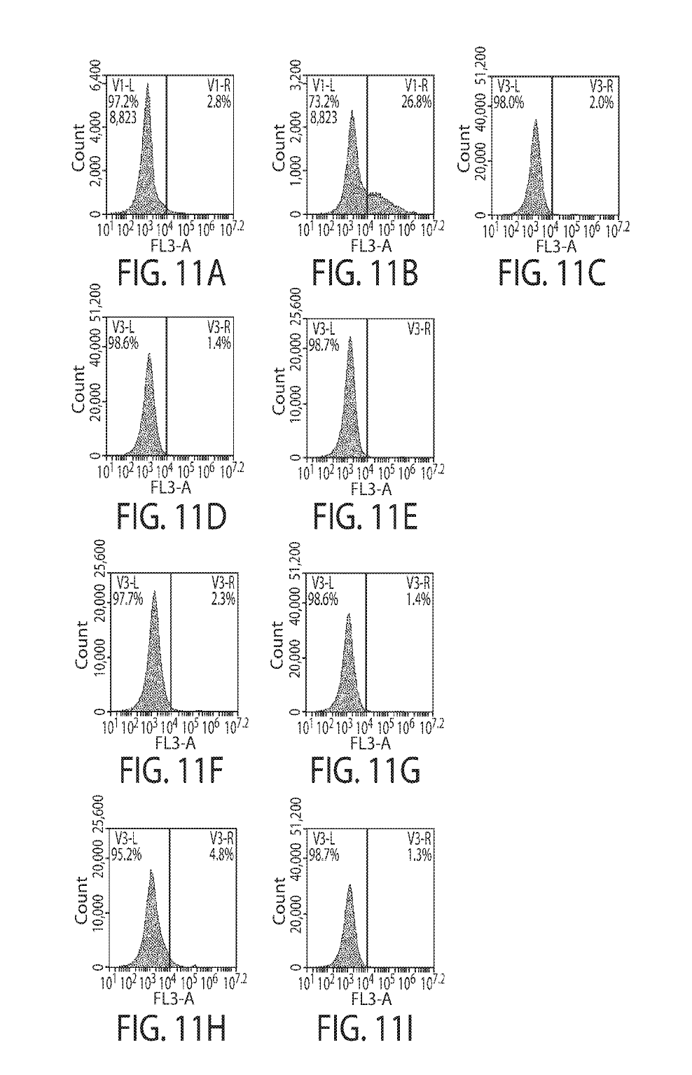

FIG. 11 shows the flow cytometry results after propidium iodide staining to NIH 3T3 mouse embryonic fibroblasts which were 3-D cultured in the peptide hydrogels prepared from the methods #1-6 and the control method following the PuraMatrix® Guidelines for Use (BD/Corning website). Specifically, FIG. 11 presents flow cytometry data of fibroblasts cultured in the 0.5% PuraMatrix hydrogels prepared from the methods #1-6. Cells were isolated from the gel using peptide hydrogel washing solution (PBS at pH 3.0). (A): Negative Control: fibroblasts harvested from cell culture flask. (B) Positive control: fibroblasts treated with 70% ethanol for 1 hour. (C) fibroblasts cultured in PuraMatrix® for 24 hours following the PuraMatrix® Guidelines for Use (BD/Corning website) (D-I) fibroblasts cultured in PuraMatrix® for 24 hours following methods #1-6, respectively. As a positive control, cells were treated with 70% ethanol for 1 hour before test and as a negative control, cells harvested from a tissue culture flask were directly tested. The results demonstrates that fibroblasts can be successfully isolated from all the hydrogels and tested for the flow cytometry analysis.

### Cell Viability:

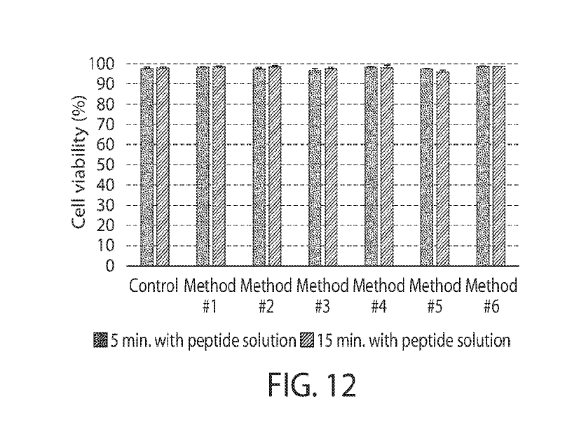

In accordance with one or more embodiments, the peptide hydrogels prepared from methods #1-6 were associated with high cell viability. A cell viability (cytotoxicity) assay was performed to measure the ability of the hydrogels prepared from the methods #1-6 to support the viability of NIH 3T3 mouse embryonic fibroblast, a frequently-used cell line in hydrogel tissue culture systems. The viability of NIH 3T3 mouse embryonic fibroblasts was analyzed from the flow cytometry results with propidium iodide staining. The cell viability analysis was shown in FIG. 12 after fibroblasts were 3-D cultured in the peptide hydrogels prepared from the methods #1-6 and the control method following the PuraMatrix® Guidelines for Use (BD/Corning website). Cells were kept with the peptide solutions for 5 min or 15 min before DMEM was added on the top of the peptide solution.

In accordance with one or more embodiments, peptide hydrogels may be transparent so that it is easy to observe 2-D or 3-D cell images. Peptide hydrogel solutions prepared with the disclosed methods are generally more viscous, so 3-D seeded cells very slowly sink down to the bottom resulting in a three-dimensionally uniform distribution. Also, gelation time is relatively fast after adding cell culture medium, so suspended cells can be evenly distributed in the hydrogels. Control PuraMarix® is less viscous and has slow gelation. Usually collagen solution is not viscous before gelation and needs a relatively long gelation time, so cells may rapidly sink down to the bottom or are more populated in the bottom area. The prepared isotonic and pH-adjusted peptide hydrogels can be mixed with drugs, cytokines, growth factors, and proteins in various embodiments to improve cell culture and drive cell growth and differentiation.

It is to be appreciated that embodiments of the methods and devices discussed herein are not limited in application to the details of construction and the arrangement of components set forth in this description or illustrated in the accompanying drawings. The methods and devices are capable of implementation in other embodiments and of being practiced or of being carried out in various ways. Examples of specific implementations are provided herein for illustrative purposes only and are not intended to be limiting. Also, the phraseology and terminology used herein is for the purpose of description and should not be regarded as limiting. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present devices and methods or their components to any one positional or spatial orientation.

Having thus described several aspects of at least one example, it is to be appreciated that various alterations, modifications, and improvements will readily occur to those skilled in the art. For instance, examples disclosed herein may also be used in other contexts. Such alterations, modifications, and improvements are intended to be part of this disclosure, and are intended to be within the scope of the examples discussed herein. Accordingly, the foregoing description is by way of example only.

What is claimed is:

1. A cell culture kit, comprising:
a synthetic peptide hydrogel solution;
a dilution solution; and
instructions to:
adjust a concentration of the synthetic peptide hydrogel solution with the dilution solution while maintaining its tonicity at a plasma osmolality level and its pH at a level of about 3.5 or less, and
conduct a cell culture experiment with the diluted synthetic peptide hydrogel solution.

2. The kit of claim 1, wherein the synthetic peptide hydrogel comprises:
RADARADARADARADA (RADA16).

3. The kit of claim 1, wherein the synthetic peptide hydrogel solution is formulated such that a pH level of the synthetic peptide hydrogel solution is about 3.5 or less, and a tonicity of the synthetic peptide hydrogel solution is at a plasma osmolality level prior to dilution.

4. The kit of claim 1, further comprising a cocktail solution formulated to adjust the pH level of the synthetic peptide hydrogel solution to about 3.5 or less, and the tonicity of the synthetic peptide hydrogel solution to the plasma osmolality level prior to dilution.

5. The kit of claim 4, wherein at least one of the cocktail solution and the dilution solution comprises one or more isotonic agents to control tonicity.

6. The kit of claim 5, wherein the one or more isotonic agents include salts, sugars, and mixtures thereof.

7. The kit of claim 3, wherein at least one of the cocktail solution and the dilution solution comprises one or more alkali salts or acidic salts to control pH.

8. The kit of claim 3, wherein the cocktail solution is hypertonic and has a pH level of between about 1 and about 14.

9. The kit of claim 1, further comprising at least one of: a source of cells to be cultured, a cell culture medium, and an isotonic buffer solution.

10. The kit of claim 9, wherein the isotonic buffer solution comprises PBS, saline, a sugar-based isotonic agent including but not limited to sucrose, or a cell culture medium including but not limited to DMEM.

11. The kit of claim 1, further comprising a washing solution.

12. The kit of claim 11, wherein the instructions further provide direction to isolate cultured cells with the washing solution, and to subject the isolated cells to cellular or molecular characterization.

13. The kit of claim 1, wherein the instructions are directed to a 3-D cell culture protocol in which the plurality of cells is suspended in buffer solution.

14. The kit of claim 1, wherein the instructions are directed to a 2-D cell culture protocol.

15. The kit of claim 3, wherein the instructions provide further direction to select the plasma osmolality level based on a target cell type and to use the cocktail solution to achieve the plasma osmolality level in the synthetic peptide hydrogel solution.

16. A cell culture method, comprising:
maintaining a pH level of a synthetic peptide hydrogel solution at about 3.5 or less;
maintaining a tonicity of the synthetic peptide hydrogel solution at a plasma osmolality level;
diluting the peptide hydrogel solution to a predetermined concentration;
culturing cells in the peptide hydrogel solution for a predetermined period of time; and
isolating the cultured cells.

17. The method of claim 16, further comprising subjecting the isolated cells to molecular characterization involving flow cytometry analysis and image observation, cell blotting, or polymerase chain reaction (PCR) testing.

18. The method of claim 16, wherein the synthetic peptide hydrogel solution comprises:
RADARADARADARADA (RADA16).

19. The method of claim 16, wherein the culturing step is directed to a 3-D cell culture experiment.

20. The method of claim 16, wherein the culturing step is directed to a 2-D cell culture experiment.

21. The method of claim 16, wherein the culturing step is directed to recruiting cells into the hydrogels from surrounding cells in vitro or in vivo.

22. The method of claim 16, wherein the culturing step is directed to an in vitro cell culture experiment.

23. The method of claim 16, wherein the culturing step is directed to an ex vivo cell culture experiment.

24. The method of claim 16, wherein the culturing step is directed to an in vivo animal or human experiment.

25. The method of claim 23, wherein the culturing step involves combination with drugs, cytokines, growth factors, peptides, and/or proteins to improve cell culture and drive cell growth and differentiation.

26. The method of claim 24, further comprising a step of selecting the plasma osmolality level based on a target species or a target cell type of cell culture experimentation.

27. The method of claim 26, wherein the tonicity is in a range of about 260 to about 360 mOsm/L.

28. A synthetic peptide hydrogel solution having a pH level of about 1.5 to about 3.5, and a tonicity at plasma osmolality in a range of about 260 to about 360 mOsm/L with respect to a target species and a target cell type.

29. The solution of claim 28, wherein the target species is selected from the group consisting of but not limited to: mouse, rat, swine, rabbit, bovine, human, insect, bacteria, and plant.

30. The solution of claim 28, wherein the target cell type is selected from the group consisting of but not limited to: fibroblast, stem cells, epithelial cells, endothelial cells, neural cells, cardiac cells, kidney cells, blood cells, muscle cells, pancreatic cell, immune cells, dendritic cells, epidermal cells, cancer cells, astrocytes, adipocytes, hepatic cells, osteoblasts, and chondrocytes.

31. The solution of claim 28, wherein the synthetic peptide hydrogel solution comprises:
RADARADARADARADA (RADA16).

32. The solution of claim 28, wherein the synthetic peptide hydrogel solution is characterized by a storage modulus of at least 5 Pa at 0.5% concentration when measured at 1 rad/sec and 1 Pa of stress.

33. The solution of claim 28, wherein the synthetic peptide hydrogel solution comprises one or more isotonic agents including salts, sugars, and mixtures thereof to control the tonicity, and one or more alkali salts or acidic salts to control the pH level.
